(19) United States
(12) Patent Application Publication
(10) Pub. No.: US 2007/0207529 A1
(43) Pub. Date: Sep. 6, 2007

(54) Production Of (S)-2-Butanol By Oxidative Racemate Resolution

(75) Inventors: Rupert Pfaller, Munich (DE); Christina Schneider, Hohenthann (DE)

Correspondence Address: BROOKS KUSHMAN P.C., 1000 TOWN CENTER, TWENTY-SECOND FLOOR, SOUTHFIELD, MI, 48075, US

(73) Assignee: WACKER CHEMIE AG, Munich (DE)

(21) Appl. No.: 11/678,177

(22) Filed: Feb. 23, 2007

(30) Foreign Application Priority Data
Mar. 2, 2006 (DE) 10 2006 009 744.0

(51) Int. Cl. C12P 7/16

(52) U.S. Cl. 435/160

(57) ABSTRACT

A method for producing (S)-2-butanol of an optical purity ee of >90% comprises reacting a biotransformation composition comprising, as starting materials, a racemic mixture of 2-butanol (rac-2-butanol), an oxidoreductase, a redox cofactor and a cosubstrate, (R)-2-butanol being selectively oxidized to 2-butanone, and (S)-2-butanol being retained in an optical purity ee of >90%.

### BACKGROUND OF THE INVENTION

1. Field of the Invention

The invention relates to a method for producing (S)-2-butanol by oxidative enzymatic resolution of a racemic mixture of 2-butanol (rac-2-butanol).

2. Background Art

Optically active hydroxyl compounds are valuable synthesis building blocks, for example in the production of pharmaceutically active compounds or agrochemicals. Therefore, they are of economic importance. The compounds can frequently only be produced with difficulty by classical chemical methods. The required optical purities for uses in the pharmaceutical or agrochemical sector can be achieved only with difficulty in this manner. Therefore, for the production of chiral compounds, biotechnological methods are being used to an increasing extent. Enzymes which can reduce carbonyl compounds, or can selectively oxidize alcohols, are increasingly being used because of their high enantioselectivity.

Enzymes of the class of oxidoreductases which are used for producing chiral compounds by reduction of prochiral carbonyl compounds are by definition designated by the collective term carbonyl reductases (hereinafter “CR”). In the majority of cases the product of a CR reaction is an alcohol. However, it is also possible that the product of a CR reaction is an amine. The enzyme classes coming under the collective term carbonyl reductases include, inter alia, alcohol dehydrogenases (hereinafter “ADH”), aldo-keto reductases, aldehyde reductases, glycerol dehydrogenases and fatty acid synthase. This broad spectrum of reducing enzymes shares the fact that they obtain the electrons for reducing carbonyl compounds from redox cofactors in their reduced form, customarily NADH or NADPH.

CRs of the ADH class are capable of reversible redox reactions; that is, certain prochiral carbonyl compounds are not reduced only to the alcohol, but the alcohol can also be oxidized to the carbonyl compound. Because of the high enantioselectivity of some ADHs, this can be exploited for oxidative racemate resolution.

The redox cofactors NAD and NADP are used stoichiometrically in oxidative racemate resolution, that is they must either be used stoichiometrically or else regenerated by reduction of a cosubstrate (termed cofactor regeneration). A cosubstrate in this case is defined as a compound which is reduced enzymatically as an oxidizing agent, with the electrons required for the reduction being transferred from NADH or NADPH, with NAD or NADP thereby being regenerated.

A chiral alcohol of great importance in synthetic chemistry is (S)-2-butanol. All current approaches for producing (S)-2-butanol share the fact that either complex derivatives must be produced for enantiomer separation (for example by enzymatic kinetic resolution, EKR, or by chemical enantiomer separation) or that in the direct enantioselective reduction of 2-butanone to (S)-2-butanol, the enantiomeric purity of (S)-2-butanol for technical applications is insufficient (for example by selective chemical or enzymatic reduction).

The simplest approach for producing (S)-2-butanol would be direct enantioselective reduction of 2-butanone, either enzymatically by an S-specific CR or by catalytic hydrogenation. Here, however, there is the problem that the methyl and ethyl groups adjacent to the prochiral carbonyl group in 2-butanone are insufficiently different sterically in order to ensure sufficient selectivity in the reduction.

WO 2005/108593 describes a method for the direct reduction of 2-butanone to (S)-2-butanol in a 2-phase system by means of a CR and a cofactor. The cofactor is regenerated by means of a cosubstrate. For the cofactor regeneration, a water-immiscible secondary alcohol in high molar excess is employed. The method is, however, associated with high production costs, since the reaction conversion rate at 68% is relatively low, and for cofactor regeneration, an expensive water-immiscible secondary alcohol, preferably 2-heptanol, is necessary. Moreover, the CR used (CR from Candida parapsilosis), is expensive. The secondary alcohol, in addition to its function as cosubstrate, because of its non-miscibility with water, must also serve for product removal during and after the reaction. Since the product (S)-2-butanol, however, is highly water soluble, in an extraction with a water-immiscible secondary alcohol, a distribution equilibrium for the product is established which leads to further significant yield losses. Overall, although (S)-2-butanol may be produced by this method in sufficiently high optical purity, it is produced with high production costs.

Stampfer et al. (2003), TETRAHEDRON ASYMMETRY 14: 275-280 described a method for oxidative racemate resolution using an S-selective ADH from Rhodococcus ruber, by which R-alcohols could be produced with high enantiomeric excess (“ee”). However, this method was not suitable for producing (R)-2-butanol from rac-2-butanol, and thus could not serve as a template for developing a similar method for production of (S)-2-butanol.

JP2004-254549 discloses a CR which oxidizes (R)-2-butanol preferentially over (S)-2-butanol to 2-butanone. The relative activity of the disclosed CR for (R)-2-butanol, however, is only higher by a factor of 2 than that for (S)-2-butanol (see Table 6 in JP2004-254549). The selectivity of the disclosed CR is thus insufficient to produce (S)-2-butanol in sufficiently high enantiomeric purity.

Therefore, conventionally, optical purities (expressed as enantiomeric excess ee) of less than 90% ee are achieved. For instance, a commercial batch of (S)-2-butanol (Aldrich) was analyzed by chiral gas chromatography (GC) and an ee of 82.2% was determined (see 1st example). In the selective enzymatic reduction of 2-butanone using an S-selective CR (enzyme T-ADH, commercially available from Jülich Chiral Solutions GmbH), (S)-2-butanol having a maximum ee of 62.5% was obtained (see 8th example).

According to the prior art, therefore, it is obviously only possible to produce (S)-2-butanol with an ee>90% efficiently with high costs or great complexity.

### SUMMARY OF THE INVENTION

It was therefore an object of the invention to provide a method which permits (S)-2-butanol to be produced on an industrial scale with an optical purity ee of >90%, preferably >93%, more preferably >97%, and most preferably greater than 99%, with simultaneously high space-time yields. These and other objects are achieved by a method which comprises reacting a biotransformation composition comprising, as starting materials, a racemic mixture of 2-butanol (rac-2-butanol), an oxidoreductase, a redox cofactor and a cosubstrate, (R)-2-butanol being selectively oxidized to 2-butanone, and (S)-2-butanol being formed in an optical purity ee of >90%.

### DETAILED DESCRIPTION OF THE PREFERRED EMBODIMENT(S)

The batch method of the invention makes possible the production of (S)-2-butanol in a high optical purity of >90%, preferably >93%, more preferably >97%, and in particular, greater than 99%, at a high space-time yield and with low enzyme usage. As shown in the Example 5, using the inventive method, (S)-2-butanol can be obtained in the course of 8-24 h with yields of 30 g/l and more, the use of enzyme-containing cells (expressed in dry biomass, see 1st example) being no more than 2 μl of biotransformation batch.

A suitable starting material is, for example, a racemic mixture of (S)-2-butanol and (R)-2-butanol (rac-2-butanol) which is obtainable commercially at favorable prices.

The oxidoreductase is preferably a CR which has R specificity.

The redox cofactor is a compound which, in its oxidized form, in a first enzymatic reaction takes up electrons from an R-specific CR, and in a second enzymatic reaction, transfers them to the cosubstrate. The electrons of the first enzymatic reaction originate in this case from the oxidation of (R)-2-butanol, present in rac-2-butanol, by the R-specific CR, with the result that after termination of the reaction, (R)-2-butanol is completely converted into 2-butanone, and (S)-2-butanol remains in the reaction mixture. The redox cofactor is preferably selected from compounds of the group NAD, NADP (in each case the oxidized form of the cofactor), NADH or NADPH (in each case the reduced form of the cofactor) and salts thereof.

The redox cofactors in their oxidized form, NAD or NADP, are consumed stoichiometrically in the oxidative racemate resolution, and must either be used stoichiometrically or else regenerated by reduction of a cosubstrate (cofactor regeneration). The stoichiometric use of NAD or NADP is not cost-effective because of the high price of these compounds. This disadvantage can be circumvented by cofactor regeneration. A precondition for this is a cheap cosubstrate (oxidizing agent) which can be reduced enzymatically by the CR. Only efficient and inexpensive regeneration of the redox cofactor makes industrial use of the oxidative racemate resolution possible.

The cosubstrate is a compound which can be reduced as an oxidizing agent enzymatically by the CR, the electrons being transferred from NADH or NADPH to the cosubstrate, and NAD or NADP thereby being regenerated.

When use is made of a CR from the class of ADHs, cofactor regeneration is possible by reduction of a cheap carbonyl compound as a cosubstrate. A suitable cosubstrate is preferably a ketone or a diketone, and most preferably acetone, 2-butanone or 2-pentanone. These cosubstrates have the property that their reduction is reversible and the alcohols formed can again be oxidized by ADH, so that in the course of the reaction a reaction equilibrium is established. Using a cosubstrate from the class of compounds which is reversibly reduced by an R-specific ADH, such as acetone, for example, (S)-2-butanol can be produced having an optical purity ee of 93% (see 4th example).

Another class of inexpensive cosubstrates includes β-ketoesters, the simplest representative being methyl acetoacetate. These compounds are reduced by ADHs to the corresponding β-hydroxy ester. In the corresponding reduction of methyl acetoacetate, methyl 3-hydroxybutyrate is formed. Cosubstrates of the class of β-ketoesters have the property that they are irreversibly reduced by ADHs to β-hydroxy esters.

It has now surprisingly been found that the combination of ADH and irreversibly reducible cosubstrate selected from the class of β-ketoesters leads to a higher enantiomeric purity of S-2-butanol in the oxidative racemate resolution of rac-2-butanol than the combination of ADH and reversibly reducible cosubstrate. Particular preference for cofactor regeneration is therefore given to a cosubstrate of the class of β-ketoesters. In particular, preferred cosubstrates are esters of acetoacetic acid, especially methyl, ethyl, isopropyl and tertiary-butyl esters.

As R-specific CRs, use is preferably made of secondary ADHs, for example from species of the genus Lactobacillus, such as the ADHs from Lactobacillus brevis (LB-ADH), Lactobacillus kefir, Lactobacillus parabuchneri, Lactobacillus kandleri or Lactobacillus minor. Preferred R-selective CRs are LB-ADH and the ADH from Lactobacillus kefir. A particularly preferred R-selective CR is LB-ADH.

The ADHs used for oxidative racemate resolution can be produced by culturing the microorganism from which the ADH in question originates. This is achieved in each case in a manner known to those skilled in the art. The ADH enzyme produced in this manner can be further used directly in the cells of the production host, or else it can be used after digestion of the cells as protein extract, or as purified protein after corresponding workup by, for example, column chromatography.

The enzyme production of the CRs can proceed using an expression system, also in recombinant form. For this, the gene coding for the CR in question is isolated and, in accordance with the prior art, cloned into an expression vector suitable for protein production. After transformation of the expression vector into a suitable host organism, a production strain is isolated. Using this production strain the CR may be produced in a manner known per se, for example by fermentation. The CR enzyme produced in this manner can then be further used directly in the cells of the production host, or else, after digestion of the cells, as protein extract, or as purified protein after appropriate workup by, for example, column chromatography.

It is known to those skilled in the art that the CR in question can be produced using a recombinant expression system with far higher yields than by culturing the original strain from which the CR originates. The recombinant production therefore makes possible a far more inexpensive production of the CR and thus contributes to improvement of the economic efficiency of the method. Preference is therefore given to the enzyme production of the CRs of the invention using an expression system in recombinant form.

For enzyme production, bacterial and eukaryotic expression systems are suitable. Host organisms for enzyme production are preferably selected from Escherichia coli, strains of the genus Bacillus, yeasts such as Pichia pastoris, Hansenula polymorpha or Saccharomyces cerevisiae and also fungi, such as Aspergillus or Neurospora, but they are not however restricted to these host organisms. The preferred expression systems include E. coli, Bacillus, Pichia pastoris, S. cerevisiae, Hansenula polymorpha or Aspergillus. Particularly preferred expression systems for production of the ADH enzyme are E. coli, Pichia pastoris and S. cerevisiae, in particular, E. coli.

To achieve enzyme usage as cost efficiently as possible, enzyme production preferably proceeds by fermentation, most preferably in a fed-batch method. Preferably, the fermenter cells are then further used directly in the method of the invention, so that the process is conducted as whole cell biotransformation. However, it is also possible to use the isolated fermenter cells, or, after digestion of the cells, to use the resultant protein extract, or, after corresponding workup by, for example, column chromatography, to use the resultant purified protein. Particular preference is given to a whole-cell biotransformation in which first the enzyme production proceeds in a recombinant host cell by means of fermentation, and the fermenter cells are subsequently used directly in a biotransformation.

A biotransformation composition according to the invention (a batch solution) comprises between 1% (v/v) and 40% (v/v), based on the total batch of fermentation medium, of fermenter cells containing a CR having a biomass fraction of 0.05-2% (w/v) and between 2% (w/v) and 50% (w/v) of the total batch of the starting material rac-2-butanol, a redox cofactor selected from the compounds NAD, NADH, NADP, NADPH and salts thereof in an amount between 10 μM and 200 μM, and between 10% (v/v) and 50% (v/v) of a cosubstrate selected from the group of compounds which are reversibly or irreversibly reduced by a CR. Preferably, the cosubstrate is one of the abovementioned compounds, and the CR is an alcohol dehydrogenase.

In a modified form of the method, one or more of the components of the biotransformation composition is added continuously or batchwise (fed-batch solution). However, the preferred method is the batch solution.

Preferably, the composition contains between 2% (v/v) and 30% (v/v) of fermentation medium containing fermenter cells having a CR having a biomass fraction of 0.1-1.5% (w/v). The biomass fraction is defined as dry biomass which is obtained when the fermenter cells are dried to constant weight, for example in a drying cabinet at 105° C. Most preferably, the composition contains between 3% (v/v) and 20% (v/v) of fermentation medium containing fermenter cells having a CR having a biomass fraction of 0.15-1% (w/v).

A biotransformation composition according to the invention is further distinguished by the fact that the fraction of starting material is between 2% (w/v) and 50% (w/v) of the total batch. Preferably, the fraction of starting material is between 3% (w/v) and 30% (w/v) of the total batch. Most preferably, the fraction of starting material is between 5% (w/v) and 20% (w/v) of the total batch.

In one embodiment, the biotransformation composition of the invention is distinguished by the fact that the fraction of cosubstrate is between 10% (v/v) and 50% (v/v). Preference is given to a composition in which the fraction of cosubstrate is between 20% (v/v) and 45% (v/v), and particular preference is given to a composition in which the fraction of cosubstrate is between 30% (v/v) and 40% (v/v).

A biotransformation composition of the invention comprises a redox cofactor selected from NAD, NADH, NADP or NADPH, or salts thereof, in an amount between 10 μM and 200 μM. Preference is given to a composition in which the fraction of redox cofactor selected from NAD, NADH, NADP or NADPH, or salts thereof, is between 15 μM and 150 μM, and particular preference is given to a composition in which the fraction of redox cofactor selected from NAD, NADH, NADP or NADPH, is between 20 μM and 80 μM.

The method is preferably carried out at a temperature of 2° C. to 50° C., preferably 3° C. to 40° C., most preferably 5° C. to 30° C., and is preferably carried out in a pH range from 5 to 10, more preferably from 6 to 9, and most preferably from 7 to 8.5. Preferably, the batch is buffered to maintain a constant pH. Preferably, pH control proceeds via a titration device, coupled to a pH meter (pH-stat method).

The reaction period of the method according to the invention is preferably 3 h to 60 h, more preferably 5 h to 40 h, and in particular 7 h to 30 h. Under these conditions, (S)-2-butanol can be produced having an ee of greater than 90%, preferably greater than 93%, more preferably greater than 97%, and most preferably greater than 99%.

By using the biotransformation compositions of the invention, it was thus possible for the first time to produce (S)-2-butanol by oxidative racemate resolution from rac-2-butanol in inexpensive form. The particular advantages of this oxidative racemate resolution are simple process procedures and the fact that no additional derivatization of (S)-2-butanol or (R)-2-butanol is required for racemate resolution.

If recovery of the enzyme is not intended, the (S)-2-butanol is obtained by direct distillation from the reaction batch or by extraction of the reaction batch after the end of the reaction, followed by a distillation. A technically simple and inexpensive method for direct distillation from the reaction batch is described in the 7th example.

In an alternative method variant, the (S)-2-butanol is obtained from the reaction batch by extraction with a solvent which is insoluble in the aqueous phase, preferably with a water-immiscible organic solvent. The extraction can proceed batchwise or continuously. Suitable organic solvents are all water-immiscible solvents which can isolate (S)-2-butanol from the aqueous phase. Preferably, use is made of organic solvents such as chlorinated hydrocarbons, esters, ethers, alkanes and aromatics. Most preferably, methylene chloride, chloroform, ethyl acetate, methyl acetate, propyl acetate, isopropyl acetate, butyl acetate, tert-butyl acetate, diethyl ether, diisopropyl ether, dibutyl ether, methyl tert-butyl ether (MTBE), pentane, hexane, heptane, toluene or mixtures thereof are employed as organic solvents. In particular, preferred solvents are methylene chloride, chloroform, MTBE and ethyl acetate.

After removal of the organic extraction phase, it is preferably worked up by distillation, enrichment of the reaction product being achieved thereby, and partial to complete removal of byproducts from the extraction solvent being effected so that the extraction solvent can be used again for extraction.

In a further alternative embodiment of the method of the invention, the CR is recovered from the reaction batch for reuse, and thus the economic efficiency of the method is again increased. In addition to high space-time yields, the repeated use of enzyme causes a low specific enzyme consumption per mole of product and thus enables higher cost efficiency of the oxidative racemate resolution according to the invention. In this method variant, the (S)-2-butanol is extracted from the reaction batch using a solvent which is insoluble in the aqueous phase, and the enzyme is isolated from the aqueous phase. A precondition for this is that the solvent does not lead to inactivation of the enzyme. Such solvents include, for example, MTBE and butyl acetate.

After removal of the organic extraction phase, it is preferably worked up by distillation, enrichment of (S)-2-butanol being achieved, and partial to complete removal of byproducts from the extraction solvent being effected for reuse of the extraction solvent.

A preferred method for isolation of (S)-2-butanol from the reaction mixture is direct distillation.

By refining the organic extraction solution containing the crude product, for example by means of fine distillation, the desired end product (S)-2-butanol is obtained. The end product (S)-2-butanol is typically distinguished by yields >30%, preferably >35%, more preferably >40%, based in each case on the amount of rac-2-butanol used. The product is further distinguished by a chemical purity>98%, preferably >99%. The product of the inventive method is distinguished by an enantiomeric excess ee>90%, preferably ee>93%, more preferably 97% or more, and in particular, greater than 99%.

The examples hereinafter serve to describe the invention, but do not limit it in any way:

### EXAMPLE 1

### Production of LB-ADH by Fermentation

The enzyme LB-ADH, its gene and the recombinant production of LB-ADH in E. coli are disclosed in EP796914. Use was made of the plasmid pADH-1 transformed into E. coli as disclosed in EP796914. Alternatively, the enzyme can be obtained commercially from Jülich Chiral Solutions GmbH as crude extract produced from recombinant E. coli.

Fermentation of LB-ADH-Producing E. coli:

### Production of an Inoculum for the Fermentation:

1. Preculture of E. coli pADH-1 in LBamp medium. Culture proceeded overnight on an orbital shaker (Infors) at 120 rpm and 30° C. LBamp medium contained peptone vegetable (Oxoid) 10 g/l; yeast extract (Oxoid) 5 g/l; NaCl 5 g/l and ampicillin 0.1 g/l.

2. Preculture: 100 ml of SM3 amp medium were inoculated with 1.3 ml of shake culture in a 1 l Erlenmeyer flask. Culture proceeded for 16-18 h at 30° C. and 120 rpm on an orbital shaker (Infors) to a cell density OD₆₀₀/ml of 7-10. 100 ml of the preculture were used to inoculate 1 l of fermenter medium.

SM3 amp medium contained peptone vegetable (Oxoid) 5 μl; yeast extract (Oxoid) 2.5 g/l; NaCl 0.1 g/l; ammonium sulfate 5 g/l; KH₂PO₄ 3 g/l; K₂HPO₄ 12 g/l; glucose 5 g/l; MgSO₄.7H₂O 0.3 g/l; CaCl₂.2H₂O 14.7 mg/l; FeSO₄.7H₂O 2 mg/l; sodium citrate.2H₂O 1 g/l; vitamin B1 5 mg/l; trace element mix 1 ml/l and ampicillin 0.1 g/l. The trace element mix had the composition H₃BO₃ 2.5 g/l; CoCl₂.6H₂O 0.7 g/l; CuSO₄.5H₂O 0.25 g/l; MnCl₂.4H₂O 1.6 g/l; ZnSO₄.7H₂O 0.3 g/l and Na₂MoO₄.2H₂O 0.15 g/l.

The fermentations were carried out in Biostat CT fermenters from Sartorius BBI Systems GmbH. Fermentation medium was FM2amp. The fermentation proceeded in fed-batch mode. FM2amp medium contained glucose 20 g/l; peptone vegetable (Oxoid) 5 g/l; yeast extract (Oxoid) 2.5 g/l; ammonium sulfate 5 g/l; NaCl 0.5 g/l; FeSO₄.7H₂O 75 mg/l; Na₃Citrate.2H₂O 1 g/l; CaCl₂.2H₂O 14.7 mg/l; MgSO₄.7H₂O 0.3 g/l; KH₂PO₄ 1.5 g/l; trace element mix 10 ml/l; vitamin B1 5 mg/l and ampicillin 0.1 g/l. The pH of the FM2amp medium was set to 7.0 before the start of fermentation.

1 l of FM2amp was inoculated with 100 ml of inoculum. Fermentation temperature was 30° C. pH of the fermentation was 7.0 and was kept constant using as correction media 25% NH₄OH, and 6 N H₃PO₄. Ventilation proceeded using compressed air at a constant flow rate of 5 slpm (standard liters per minute). The oxygen partial pressure pO₂ was set to 50% saturation. The oxygen partial pressure was controlled via the stirrer speed (stirrer speed 450-1300 rpm). To control foam formation, Struktol J673 (20-25% v/v in water) was used.

In the course of the fermentation, the glucose consumption was determined by off-line glucose measurement using a glucose analyzer from YSI. As soon as the glucose concentration of the fermentation batches was approximately 5 g/l (5-6 h after inoculation), the addition of a 60% w/w glucose feed solution was started. The flow rate of the feed was selected in such a manner that during the production phase a glucose concentration of 1-5 g/l could be maintained.

LB-ADH production was induced by adding IPTG (stock solution 100 mM) at a concentration of 0.4-0.8 mM, as soon as cell growth in a fermenter had reached an OD₆₀₀/ml of 50-60. The entire fermentation period was 32 h. After termination of fermentation, the fermenter broth was frozen in aliquots each of 100 ml. By drying of aliquots of fermenter cells in a drying cabinet at 105° C. to constant weight, the dry biomass was determined. It was 50 g/l of fermenter broth.

### EXAMPLE 2

### Production of an LB-ADH Crude Extract

2 l of cell suspension from the fermentation of E. coli pADH-1 (see 1st example) were centrifuged (15 min. 8000 rpm at 4° C., GS 3 rotor, Sorvall centrifuge). The sediment was resuspended in 500 ml of 50 mM potassium phosphate, pH 7.0, 1 mM MgCl₂ and disintegrated by three passages through a high-pressure homogenizer (NS1001L Panda 2K from Niro Soavi) at 800 bar pressure. The homogenate was centrifuged (30 min. 8000 rpm at 4° C., GS 3 rotor, Sorvall centrifuge). The supernatant gave an LB-ADH crude extract of 535 ml volume. The LB-ADH activity determination gave a volume activity of 1300 U/ml, or a specific activity of 108 U/mg of protein in the crude extract.

### Spectrophotometric Determination of LB-ADH Activity:

The measurement batch of 1 ml volume for the photometric determination of LB-ADH activity was composed of measurement buffer (0.1 M potassium phosphate, pH 7.0, 0.1 M NaCl, 1 mM MgCl₂), 3 μl of substrate ethyl 4-Cl-acetoacetate, 0.2 mM NADPH and LB-ADH-containing cell extract. Measurement temperature was 25° C. The reaction was started by adding LB-ADH cell extract and the decrease in extinction owing to consumption of NADPH at a wavelength of 340 nm was measured (extinction coefficient of NADPH: ε=0.63×10⁴ l×Mol⁻¹×cm⁻¹). One unit of LB-ADH activity is defined as the consumption of 1 μmol of NADPH/min. under test conditions.

To determine the specific activity, the protein concentration of the cell extracts was determined in a manner known per se using the “BioRad Proteinassay” from BioRad.

### EXAMPLE 3

### Gas Chromatographic Analysis

Reference substances for (R)- and (S)-2-butanol and also methyl ethyl ketone are commercially available (Aldrich).

### Chiral GC:

A gas chromatograph 6890N from Agilent described in the prior art, including a flame-ionization detector, and fitted with a CP-Chirasil-Dex-CB column from Varian (25 m×0.25 mm) was used for chiral separation. For the gas chromatographic separation, a temperature gradient of 40° C.-170° C. having a gradient slope of 20° C./min. was set. Retention times under these conditions were:

Methyl ethyl ketone: 5.5 min.

### (R)-2-Butanol: 14.1 min.

### (S)-2-Butanol: 14.4 min.

For the reference substances, the enantiomeric excess ee was determined. For (R)-2-butanol, the ee was 86.8%, for (S)-2-butanol, the ee was 82.2%.

### EXAMPLE 4

### Oxidative Racemate Resolution of rac-2-butanol Using LB-ADH Cells and Acetone as Cosubstrate

Batch 1: A reaction batch was composed of 5 ml (4 g) of rac-2-butanol, 40 ml of acetone, 1.2 ml of LB-ADH cells, 50 μM NADP and 53.8 ml of KPi buffer. The composition of KPi buffer was 0.1 M potassium phosphate, pH 8.0, 0.1 M NaCl, 1 mM MgCl₂. The reaction batch was stirred at 15° C. At various time points, 0.1 ml samples of the reaction batch were taken, extracted with 0.9 ml of methylene chloride and analyzed by chiral GC (see 3rd example). The reaction course is shown in Table 1. For a reaction temperature of 15° C. and a pH of 8.0, the enantiomeric excess ee of the product (S)-2-butanol was 92.6%.

TABLE 1

| Time | Methyl ethyl | (R)-2- | (S)- | |
|---|---|---|---|---|
| (h) | ketone (%) | butanol (%) | 2-butanol (%) | ee (S)-2-butanol (%) |
| 0 | 0 | 49.9 | 50.1 | 0.2 |
| 0.5 | 10.64 | 37.09 | 47.89 | 12.8 |
| 1 | 18.31 | 30.14 | 48.00 | 22.8 |
| 1.7 | 23.04 | 25.51 | 48.15 | 30.8 |
| 2.4 | 29.13 | 19.65 | 48.01 | 42 |
| 3.6 | 34.24 | 14.49 | 47.73 | 53.4 |
| 4.3 | 38.64 | 11.76 | 49.6 | 61.6 |
| 5.4 | 40.69 | 8.15 | 47.82 | 70.8 |
| 5.9 | 41.36 | 7.2 | 47.52 | 73.6 |
| 23.1 | 51.29 | 1.67 | 43.9 | 92.6 |

Batch 2: A reaction batch was composed of 5 ml (4 g) of rac-2-butanol, 40 ml of acetone, 1.2 ml of LB-ADH cells, 50 μM NADP and 53.8 ml of KPi buffer. The composition of KPi buffer was 0.1 M potassium phosphate, pH 8.0, 0.1 M NaCl, 1 mM MgCl₂. The reaction batch was stirred at 5° C. At various time points, 0.1 ml samples of the reaction batch were taken, extracted with 0.9 ml of methylene chloride and analyzed by chiral GC (see 3rd example). The course of the reaction is shown in Table 2. For a reaction temperature of 5° C. and a pH of 8.0, the enantiomeric excess ee of the product (S)-2-butanol was 93.4%.

TABLE 2

| Time | Methyl ethyl | (R)-2- | (S)- | |
|---|---|---|---|---|
| (h) | ketone (%) | butanol (%) | 2-butanol (%) | ee (S)-2-butanol (%) |
| 0 | 0 | 49.8 | 50.2 | 0.4 |
| 0.2 | 16.11 | 34.32 | 48.95 | 17.6 |
| 2 | 24.98 | 25.37 | 49.25 | 32.0 |
| 4 | 37.87 | 11.77 | 47.90 | 60.5 |
| 6 | 42.92 | 6.60 | 47.76 | 75.7 |
| 24 | 51.94 | 1.44 | 42.39 | 93.4 |

Batch 3: A reaction batch was composed of 5 ml (4 g) of rac-2-butanol, 40 ml of acetone, 1.2 ml of LB-ADH cells, 50 μM NADP and 53.8 ml of KPi buffer. The composition of KPi buffer was 0.1 M potassium phosphate, pH 9.0, 0.1 M NaCl, 1 mM MgCl₂. The reaction batch was stirred at 5° C. At various time points, 0.1 ml samples of the reaction batch were taken, extracted with 0.9 ml of methylene chloride and analyzed by chiral GC (see 3rd example). The course of the reaction is shown in Table 3. For a reaction temperature of 5° C. and a pH of 9.0, the enantiomeric excess ee of the product (S)-2-butanol was 92.8%.

TABLE 3

| Time | Methyl ethyl | | | ee (S)-2- |
|---|---|---|---|---|
| (h) | ketone (%) | (R)-2-butanol (%) | (S)-2-butanol (%) | butanol (%) |
| 0 | 0 | 49.7 | 50.3 | 0.6 |
| 0.2 | 14.03 | 36.08 | 49.26 | 15.4 |
| 1.8 | 32.56 | 16.92 | 48.64 | 48.4 |
| 16 | 50.47 | 1.82 | 45.72 | 92.4 |
| 19.9 | 50.22 | 1.71 | 45.00 | 92.6 |
| 21.1 | 50.95 | 1.70 | 45.51 | 92.8 |

### EXAMPLE 5

### Oxidative Racemate Resolution of rac-2-butanol Using LB-ADH Cells and Methyl Acetoacetate as Cosubstrate

Batch 1: A reaction batch was composed of 5 ml (4 g) of rac-2-butanol, 20 ml of methyl acetoacetate, 1.2 ml of LB-ADH cells, 50 μM NADP and 73.8 ml of KPi buffer. The composition of KPi buffer was 0.1 M potassium phosphate, pH 8.5, 0.1 M NaCl, 1 mM MgCl₂. The reaction batch was stirred at 5° C. At various time points, 0.1 ml samples of the reaction batch were taken, extracted with 0.9 ml of methylene chloride and analyzed by chiral GC (see 3rd example). The course of the reaction is shown in Table 4. For a reaction temperature of 5° C. and a pH of 8.5, the enantiomeric excess ee of the product (S)-2-butanol was 98.1%.

TABLE 4

| Time | Methyl ethyl | | | ee (S)-2- |
|---|---|---|---|---|
| (h) | ketone (%) | (R)-2-butanol (%) | (S)-2-butanol (%) | butanol (%) |
| 0 | 0 | 50.1 | 49.9 | −0.2 |
| 1 | 16.36 | 33.85 | 45.73 | 14.9 |
| 2 | 23.74 | 27.82 | 44.77 | 23.4 |
| 19.3 | 56.04 | 1.00 | 38.87 | 95.0 |
| 23.6 | 56.98 | 0.60 | 38.51 | 96.9 |
| 26.4 | 57.67 | 0.50 | 38.05 | 97.4 |
| 40.5 | 60.64 | 0.42 | 35.76 | 97.7 |
| 48.1 | 63.25 | 0.34 | 35.66 | 98.1 |

Batch 2: A reaction batch was composed of 5 ml (4 g) of rac-2-butanol, 20 ml of methyl acetoacetate, 2.4 ml of LB-ADH cells, 50 μM NADP and 72.6 ml of KPi buffer. The composition of KPi buffer was 0.1 M potassium phosphate, pH 8.5, 0.1 M NaCl, 1 mM MgCl₂. The reaction batch was stirred at 5° C. At various time points, 0.1 ml samples of the reaction batch were taken, extracted with 0.9 ml of methylene chloride and analyzed by chiral GC (see 3rd example). The course of the reaction is shown in Table 5. For a reaction temperature of 5° C. and a pH of 8.5, the enantiomeric excess ee of the product (S)-2-butanol was 98.0%.

TABLE 5

| Time | Methyl ethyl | | | ee (S)-2- |
|---|---|---|---|---|
| (h) | ketone (%) | (R)-2-butanol (%) | (S)-2-butanol (%) | butanol (%) |
| 0 | 0 | 49.9 | 50.1 | 0.2 |
| 0.2 | 21.43 | 31.14 | 47.43 | 20.7 |
| 1.6 | 32.46 | 21.35 | 46.19 | 36.8 |
| 3.6 | 48.46 | 8.00 | 43.54 | 69.0 |
| 5 | 50.20 | 6.60 | 43.20 | 73.5 |
| 6.5 | 54.10 | 3.55 | 42.35 | 84.5 |
| 21.7 | 62.75 | 0.46 | 36.60 | 97.5 |
| 25.2 | 63.09 | 0.36 | 35.76 | 98.0 |

Batch 3: A reaction batch was composed of 10 ml (8.1 g) of rac-2-butanol, 40 ml of methyl acetoacetate, 4 ml of LB-ADH cells, 50 μM NADP and 46 ml of KPi buffer. The composition of KPi buffer was 0.1 M potassium phosphate, pH 8.5, 0.1 M NaCl, 1 mM MgCl₂. The reaction batch was stirred at 5° C. At various time points, 0.1 ml samples of the reaction batch were taken, extracted with 0.9 ml of methylene chloride and analyzed by chiral GC (see 3rd example). The course of the reaction is shown in Table 6. For a reaction temperature of 5° C. and a pH of 8.5, the enantiomeric excess ee of the product (S)-2-butanol was 98.2%.

TABLE 6

| Time | Methyl ethyl | | | ee (S)-2- |
|---|---|---|---|---|
| (h) | ketone (%) | (R)-2-butanol (%) | (S)-2-butanol (%) | butanol (%) |
| 0 | 0 | 49.80 | 50.20 | 0.4 |
| 0.2 | 18.72 | 34.31 | 46.97 | 15.6 |
| 2.4 | 33.31 | 18.89 | 43.87 | 39.8 |
| 4.2 | 41.91 | 11.32 | 42.40 | 57.9 |
| 5.2 | 46.89 | 7.74 | 41.42 | 68.5 |
| 6 | 49.02 | 6.00 | 40.24 | 74.0 |
| 7.5 | 50.79 | 3.66 | 39.47 | 83.0 |
| 24.6 | 62.44 | 0.32 | 34.21 | 98.0 |
| 27.4 | 61.88 | 0.30 | 33.79 | 98.2 |

Batch 4: A reaction batch was composed of 10 ml (8.1 g) of rac-2-butanol, 40 ml of methyl acetoacetate, 4 ml of LB-ADH cells, 50 μM NADP and 46 ml of KPi buffer. The composition of KPi buffer was 0.1 M potassium phosphate, pH 8.0, 0.1 M NaCl, 1 mM MgCl₂. The reaction batch was stirred at 15° C. At various time points, 0.1 ml samples of the reaction batch were taken, extracted with 0.9 ml of methylene chloride and analyzed by chiral GC (see 3rd example). The course of the reaction is shown in Table 7. For a reaction temperature of 15° C. and a pH of 8.0, the enantiomeric excess ee of the product (S)-2-butanol after 8 h of reaction time was 98.0%. The content of (S)-2-butanol in the reaction mixture was 30 g/l.

TABLE 7

| Time | Methyl ethyl | | | ee (S)-2- |
|---|---|---|---|---|
| (h) | ketone (%) | (R)-2-butanol (%) | (S)-2-butanol (%) | butanol (%) |
| 0 | 0 | 49.83 | 50.17 | 0.2 |
| 0.2 | 16.00 | 32.40 | 57.60 | 25.2 |
| 1 | 41.46 | 12.99 | 45.55 | 55.6 |
| 2 | 51.43 | 5.00 | 43.57 | 79.4 |
| 3 | 55.23 | 2.40 | 42.37 | 89.2 |
| 4 | 57.14 | 1.11 | 41.55 | 94.8 |
| 5 | 57.96 | 0.71 | 41.12 | 96.6 |
| 6 | 59.21 | 0.55 | 40.03 | 97.3 |
| 7 | 59.94 | 0.48 | 39.39 | 97.6 |
| 8 | 60.99 | 0.39 | 38.41 | 98.0 |

### EXAMPLE 6

### Oxidative Racemate Resolution of rac-2-butanol Using LB-ADH Enzyme and Methyl Acetoacetate as Cosubstrate

A reaction batch was composed of 10 ml of rac-2-butanol, 40 ml of methyl acetoacetate, 4 ml of LB-ADH enzyme (5200 U, see 2nd example), 50 μM NADP and 46 ml of KPi buffer. The composition of KPi buffer was 0.1 M potassium phosphate, pH 8.5, 0.1 M NaCl, 1 mM MgCl₂. The reaction batch was stirred at 5° C. At various times points, 0.1 ml samples of the reaction batch were taken, extracted with 0.9 ml of methylene chloride and analyzed by chiral GC (see 3rd example). The course of the reaction is shown in Table 8. For a reaction temperature of 5° C. and a pH of 8.5, the enantiomeric excess ee of the product (S)-2-butanol was 97.9%.

TABLE 8

| Time | Methyl ethyl | | | ee (S)-2- |
|---|---|---|---|---|
| (h) | ketone (%) | (R)-2-butanol (%) | (S)-2-butanol (%) | butanol (%) |
| 0 | 0 | 49.8 | 50.2 | 0.4 |
| 0.2 | 16.26 | 34.26 | 45.78 | 14.4 |
| 1.2 | 21.79 | 28.99 | 45.30 | 22.0 |
| 2.5 | 33.95 | 19.05 | 43.64 | 39.2 |
| 4.1 | 42.29 | 11.43 | 42.60 | 57.7 |
| 5.4 | 47.99 | 7.05 | 41.53 | 71.0 |
| 20.7 | 57.73 | 0.59 | 38.55 | 97.0 |
| 24.5 | 57.26 | 0.41 | 38.45 | 97.9 |

### EXAMPLE 7

### Production of (S)-2-butanol on a Pilot Scale

The batch (950 l end volume) was composed of 380 l of methyl acetoacetate (406.6 kg), 94.6 l of rac-2-butanol (76.6 kg), 9 l of 10× buffer (0.2 M potassium phosphate, pH 8.5, 0.2 M NaCl, 10 mM MgCl₂), 457.7 l of water, 18.9 g of NADP disodium salt (25 μM end concentration) and 10 l of LB-ADH cells. The reaction batch was stirred at 15° C. At various time points, 0.1 ml samples of the reaction batch were taken, extracted with 0.9 ml of methylene chloride and analyzed by chiral GC (see 3rd example). After 16.5 h, the reaction was stopped by acidifying with phosphoric acid to pH 3.0, briefly heating to 72° C., followed by neutralization with NaOH to pH 6.8.

At a bottom temperature of 94-96° C., the product was distilled off in a mixture with 2-butanone and water. By azeotropic distillation with MTBE, the water was removed from the crude fraction and 52.3 kg of a 90.5% (S)-2-butanol crude fraction were obtained. By further distillation of this crude fraction (5 m steel column from Montz, bottom temperature 98-115° C.), 32.6 kg (S)-2-butanol (42.6% yield based on the amount of rac-2-butanol used) having an ee of 98% and a purity of 99.2% were obtained.

### EXAMPLE 8 (COMPARATIVE EXAMPLE)

### Reduction of Methyl Ethyl Ketone to (S)-2-butanol Using T-ADH Enzyme

One reaction batch was composed of 5 ml of methyl ethyl ketone, 45 ml of isopropanol, 44 ml of KPi buffer, pH 7.0, 6 ml of T-ADH enzyme (1200 U of T-ADH, obtained from Jülich Chiral Solutions GmbH) and 50 μM NADP. The reaction batch was stirred at 30° C. At various time points, 0.1 ml samples of the reaction batch were taken, extracted with 0.9 ml of methylene chloride and analyzed by chiral GC (see 3rd example). The course of the reaction is shown in Table 9. For a reaction temperature of 30° C. and a pH of 7.0, the enantiomeric excess ee of the product (S)-2-butanol was 8.8%.

TABLE 9

| Time | Methyl ethyl | | | ee (S)-2- |
|---|---|---|---|---|
| (h) | ketone (%) | (R)-2-butanol (%) | (S)-2-butanol (%) | butanol (%) |
| 0 | 100 | 0 | 0 | 0 |
| 0.25 | 85.76 | 1.91 | 8.28 | 62.5 |
| 2 | 28.20 | 15.82 | 51.93 | 53.3 |
| 4 | 12.34 | 23.65 | 60.19 | 43.6 |
| 20 | 9.10 | 40.13 | 49.80 | 10.8 |
| 24 | 8.90 | 41.52 | 49.58 | 8.8 |

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A method for producing (S)-2-butanol of an optical purity ee of >90%, comprising reacting a biotransformation composition comprising, as starting materials, a racemic mixture of 2-butanol, an oxidoreductase, a redox cofactor and a cosubstrate, (R)-2-butanol being selectively oxidized to 2-butanone, and (S)-2-butanol being retained in an optical purity ee of >90%.

2. The method of claim 1, wherein the oxidoreductase is a carbonyl reductase having R specificity.

3. The method of claim 2, wherein, as an R-specific carbonyl reductase, a secondary ADH is employed.

4. The method of claim 2, wherein, as an R-specific carbonyl reductase, an LB-ADH is employed.

5. The method of claim 1, wherein a redox cofactor comprises at least one of NAD, NADP, NADH, NADPH, or salts thereof.

6. The method of claim 1, wherein the cosubstrate is a compound which can be reduced, as an oxidizing agent, enzymatically by the oxidoreductase, electrons being transferred from NADH or NADPH to the cosubstrate, and NAD or NADP being thereby regenerated.

7. The method of claim 1, wherein the carbonyl reductase is an alcohol dehydrogenase and the cosubstrate is a compound which is reversibly reduced by an alcohol dehydrogenase.

8. The method of claim 7, wherein the cosubstrate is a ketone, diketone or mixtures thereof.

9. The method of claim 7, wherein the cosubstrate comprises acetone, 2-butanone or 2-pentanone or a mixture thereof.

10. The method of claim 1, wherein the carbonyl reductase is an alcohol dehydrogenase, and the cosubstrate is a β-ketoester.

11. The method of claim 10, wherein the cosubstrate is an acetoacetate.

12. The method of claim 10, wherein the substrate comprises at least one acetoacetate selected from the group consisting of methyl acetoacetate, ethyl acetoacetate, isopropyl acetoacetate, and t-butyl acetoacetate.

13. A biotransformation composition suitable for use in the method of claim 1, comprising between 1% (v/v) and 40% (v/v), based on the total batch of fermentation medium, of fermenter cells containing a CR having a biomass fraction of 0.05-2% (w/v) and between 2% (w/v) and 50% (w/v) of the total batch of the starting material rac-2-butanol, a redox cofactor selected from the compounds NAD, NADH, NADP, NADPH and salts thereof in an amount between 10 μM and 200 μM, and between 10% (v/v) and 50% (v/v) of a cosubstrate selected from the group of compounds which are reversibly or irreversibly reduced by a CR.

14. The method of claim 1, carried out at a temperature of 2° C. to 50° C.

15. The method of claim 1, carried out at a temperature of 3° C. to 40° C.

16. The method of claim 1, carried out in a pH range of from 5 to 10.

17. The method of claim 1, carried out in a pH range of from 6 to 9.

18. The method of claim 1, carried out over a time period of 3 h to 60 h.

19. The method of claim 1, carried out over a time period of 5 h to 40 h.

20. The method of claim 1, wherein (S)-2-butanol is extracted from the reaction batch by means of a water-immiscible organic solvent, or is separated by distillation.
